(19) United States
(12) Plant Patent Application Publication
(10) Pub. No.: US 2008/0320625 P1
(43) Pub. Date: Dec. 25, 2008

(54) STRAWBERRY PLANT NAMED 'DRISSTRAWTHREE'

(76) Inventors: Michael D. Ferguson, Moorpark, CA (US); Bruce D. Mowrey, Watsonville, CA (US)

Correspondence Address: JONDLE & ASSOCIATES P.C., 858 HAPPY CANYON ROAD SUITE 230, CASTLE ROCK, CO, 80108, US

(21) Appl. No.: 11/821,016

(22) Filed: Jun. 21, 2007

(51) Int. Cl. A01H 5/00

(52) U.S. Cl. PLT/208

(57) ABSTRACT

This invention relates to a new and distinct cultivar of strawberry plant named ‘DrisStrawThree’. The new cultivar is primarily characterized by its large fruit size, heavy fruit production, and resistance to powdery mildew.

### GENUS AND SPECIES

Fragaria×ananassa

### VARIETY DENOMINATION

‘DrisStrawThree’

### BACKGROUND OF THE NEW PLANT

The present invention relates to a new and distinct strawberry cultivar designated ‘DrisStrawThree’ and botanically known as Fragaria×ananassa. This new strawberry cultivar was discovered in October, 2002 and originated from a cross between the female parent ‘5F205’, a proprietary strawberry plant (unpatented) and the male parent ‘San Juan’ (U.S. Plant Pat. No. 12,899). The original seedling of the new cultivar was asexually propagated at a nursery in Shasta County, California. ‘DrisStrawThree’ was subsequently asexually propagated and underwent further testing at a nursery in Ventura County, California for four years. The present invention has been found to retain its distinctive characteristics through successive asexual propagations.

### DESCRIPTION OF THE PHOTOGRAPHS

The accompanying color photographs show typical specimens of the new cultivar at various stages of development as nearly true as it is possible to make in color reproductions.

FIG. 1 shows overall plant habit including fruit at various stages of development.

FIG. 2 shows leaves of the plant with three leaflets.

FIG. 3 shows both the upperside and underside of several of the flowers.

FIG. 4 shows the whole fruit.

FIG. 5 shows the fruit in longitudinal cross-section.

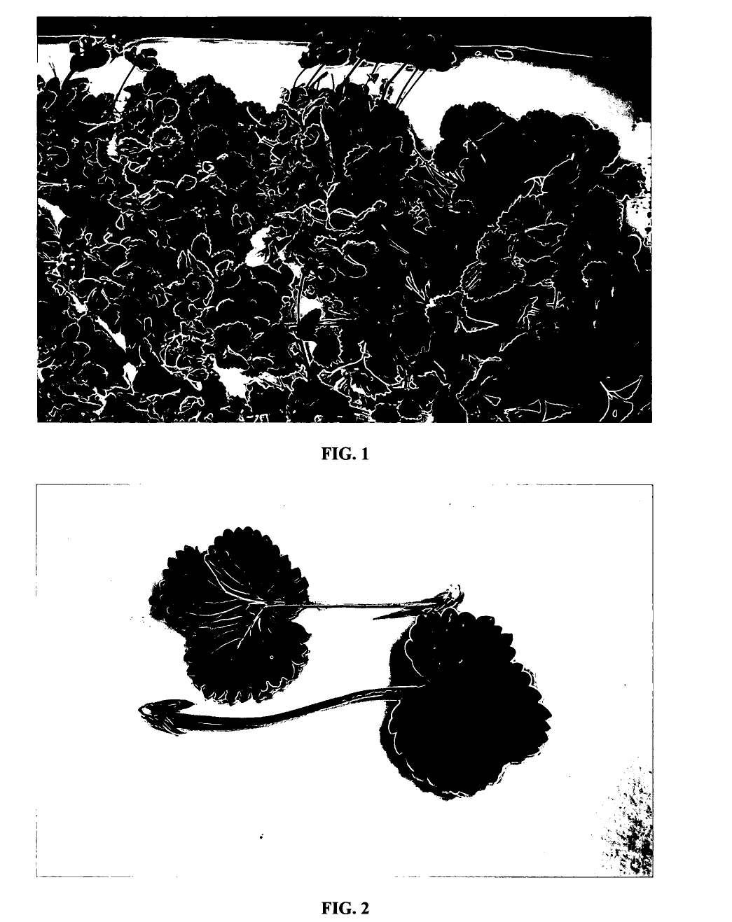

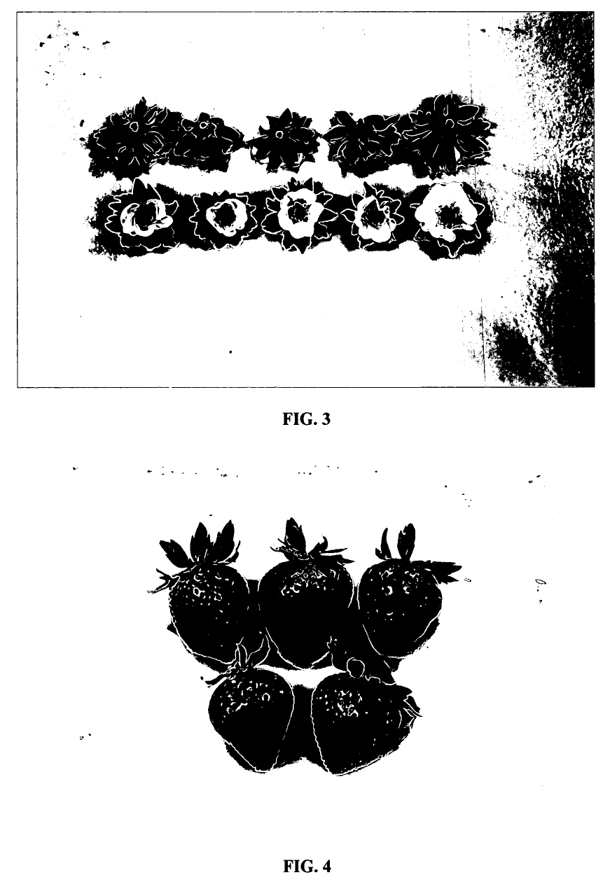

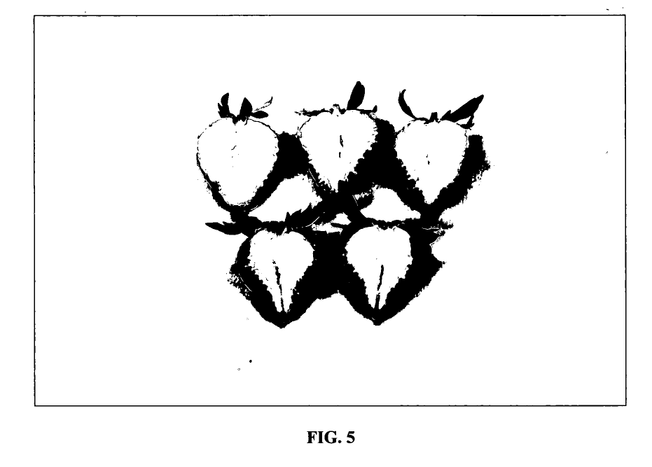

### DESCRIPTION OF THE NEW CULTIVAR

The following description of ‘DrisStrawThree’ is based on observations taken from the 2002 to 2006 growing seasons in Ventura County, California. This description is in accordance with UPOV terminology. Color designations, color descriptions, and other phenotypical descriptions may deviate from the stated values and descriptions depending upon variation in environmental, seasonal, climatic and cultural conditions. ‘DrisStrawThree’ has not been observed under all possible environmental conditions. Color terminology follows The Royal Horticultural Society Colour Chart, London (R.H.S.) (2001).

### DETAILED BOTANICAL DESCRIPTION

Table 1 shows plant characteristics of the new variety compared with plant characteristics of the parent ‘San Juan’. Plant characteristics include plant height, leaf shape in cross section, fruit hollow center, fruit texture when tasted, and reaction to aphids.

TABLE 1

| Characteristic | ‘DrisStrawThree’ | ‘San Juan’ |
|---|---|---|
| Plant height (cm) | 20.5 | 28.0 |
| Leaf shape in | Slightly concave | Flat to slightly |
| cross | | convex |
| Fruit hollow center | Small | Medium |
| Fruit texture when | Very fine | Medium |
| tasted | | |
| Aphis spp. | Moderately | Susceptible |
| (Aphids) | resistant | |

Table 2 shows plant characteristics of the new variety compared with plant characteristics of the commercial varieties, ‘Driscoll Camarillo’ (U.S. Plant Pat. No. 14,771) and ‘Baeza’ (U.S. Plant Pat. No. 11,548). Plant characteristics include plant height, diameter, number of crowns per plant, habit, density of individual plant and vigor.

TABLE 2

| | | ‘Driscoll | |
|---|---|---|---|
| Characteristic | ‘DrisStrawThree’ | Camarillo’ | ‘Baeza’ |
| Plant height (cm) | 20.5 | 23.3 | 22.4 |
| Plant diameter | 45.1 | 43.9 | 44.7 |
| (cm) | | | |
| Number of | 4 | 3 | 3 |
| crowns/plant | | | |
| Habit | Flat globose | Globose | Globose |
| Density of | Dense | Medium to | Medium to |
| individual plant | Dense | Dense | |
| Vigor | Strong | Strong | Medium |

Table 3 shows leaf characteristics of the new cultivar compared with leaf characteristics of ‘Driscoll Camarillo’ and ‘Baeza’. Leaf characteristics include terminal leaflet length and width in centimeters, length to width ratio, number of teeth per terminal leaflet, shape of teeth, color of upperside and underside of leaf, leaf shape in cross section, leaf blistering, leaf glossiness, number of leaflets, leaflet margin, and shape of leaf base.

TABLE 3

| | | ‘Driscol | |
|---|---|---|---|
| Leaf Characteristic | ‘DrisStrawThree’ | Camarillo’ | ‘Baeza’ |
| Terminal leaflet | 0.82 | 0.90 | 0.83 |
| length (cm) | | | |
| Terminal leaflet | 0.80 | 0.97 | 0.91 |
| width (cm) | | | |
| Terminal leaflet | 1.04 | 0.93 | 0.91 |
| length/width ratio | | | |
| No. teeth/terminal | 17 | 27 | 29 |
| leaflet | | | |
| Shape of teeth | Rounded | Rounded | Rounded |
| Color of upperside | RHS 139A | RHS N189B | RHS 147A |
| of leaf | | | |
| Color of underside | RHS 191B | RHS 137C | RHS 137C |
| of leaf | | | |
| Leaf shape in cross | Slightly concave | Slightly concave | Concave |
| section | | | |
| Leaf blistering | Weak | Medium | Medium |
| Leaf glossiness | Weak | Weak | Weak |
| No. leaflets | Three only | Three only | Three only |
| Terminal leaflet | Revolute | Revolute | Revolute |
| margin | | | |
| Terminal leaflet | Obtuse | Rounded | Rounded |
| base shape | | | |

Table 4 shows information about the petiole, the petiolule, the bract, and the stipule of the new cultivar compared to ‘Driscoll Camarillo’ and ‘Baeza’. This includes petiole length in centimeters, petiole diameter in centimeters, petiolule length in centimeters, petiolule diameter in centimeters, bract frequency per petiole, stipule length in centimeters, stipule width in centimeters, stipule pubescence, petiole pubescence, pose of hairs on the petiole, color of the petiole and color of the petiolule.

TABLE 4

| Characteristic | ‘DrisStrawThree’ | ‘Driscoll Camarillo’ | ‘Baeza’ |
|---|---|---|---|
| Petiole length (cm) | 14.1 | 12.7 | 13.4 |
| Petiole diameter | 0.412 | 0.408 | 0.420 |
| (cm) | | | |
| Petiole | Dense | Sparse | Medium |
| pubescence | | | to dense |
| Petiole pose of | Outward to | Outwards | Outwards |
| hairs | downward | | |
| Petiole color | RHS 144B | RHS 144B | RHS 144B |
| Petiolule color | RHS 145B | RHS 145B | RHS 145B |
| Petiolule length | 1.098 | 1.255 | 1.456 |
| (cm) | | | |
| Petiolule diameter | 0.236 | 0.254 | 0.232 |
| (cm) | | | |
| Bract frequency | 1 | 1 | 1 |
| Stipule length (cm) | 3.8 | 4.0 | 3.7 |
| Stipule width (cm) | 0.985 | 1.070 | 1.256 |
| Stipule | Dense | Sparse | Sparse |
| pubescence | | | |

Table 5 shows stolon characteristics of the new cultivar compared to ‘Driscoll Camarillo’ and ‘Baeza’. These characteristics include the number of stolons, the anthocyanin coloration of the stolons, the thickness of the stolons, and the pubescence of the stolons.

TABLE 5

| | | ‘Driscoll | |
|---|---|---|---|
| Characteristic | ‘DrisStrawThree’ | Camarillo’ | ‘Baeza’ |
| Stolon Number | Few to medium | Few to medium | Few to |
| | | | medium |
| Stolon Anthocyanin | Strong | Weak to medium | Weak to |
| | | | medium |
| Stolon Thickness | Medium | Thick | Thick to |
| | | | very thick |
| Stolon Pubescence | Sparse | Sparse | Medium |
| | | | to dense |

Table 6 shows inflorescence characteristics of the new cultivar compared to ‘Driscoll Camarillo’ and ‘Baeza’. These characteristics include inflorescence position relative to foliage, relative flower size, flower diameter in centimeters (measured from petal tip to petal tip), relative spacing of petals, petal length in centimeters, petal width in centimeters, petal length to width ratio, petal color, calyx diameter in centimeters (measured on back of flower from sepal tip to sepal tip), diameter of calyx relative to corolla, diameter of inner calyx relative to outer, sepal length in centimeters (measured from sepal tip to point of attachment to receptacle), sepal width in centimeters, receptacle color and anther color.

TABLE 6

| | | ‘Driscoll | |
|---|---|---|---|
| Characteristic | ‘DrisStrawThree’ | Camarillo’ | ‘Baeza’ |
| Inflorescence position | Beneath | Above | Above |
| relative to foliage | | | |
| Flower size | Medium | Medium | Medium |
| Flower diameter (cm) | 3.001 | 2.263 | 2.490 |
| Petal spacing | Overlapping | Overlapping | Overlapping |
| Petal length (cm) | 1.502 | 1.262 | 1.496 |
| Petal width (cm) | 1.531 | 1.263 | 1.511 |
| Petal length/width ratio | 0.98 | 1.00 | 0.99 |
| Petal color | RHS 155B | RHS 155C | RHS 155B |
| Calyx diameter (cm) | 4.088 | 2.550 | 2.925 |
| Calyx diameter relative | Larger | Smaller | Same size |
| to corolla | | | |
| Inner calyx diameter | Same size | Same size | Same size |
| relative to outer | | | |
| Sepal length (cm) | 1.582 | 0.964 | 1.087 |
| Sepal width (cm) | 0.817 | 0.544 | 0.505 |
| Receptacle color | RHS N144A | RHS 2B | RHS 1A |
| Anther color | RHS 17A | RHS 15B | RHS 17B |

Table 7 shows fruit characteristics of the new cultivar compared to ‘Driscoll Camarillo’ and ‘Baeza’.

TABLE 7

| | | ‘Driscoll | |
|---|---|---|---|
| Characteristic | ‘DrisStrawThree’ | Camarillo’ | ‘Baeza’ |
| Fruiting truss | 13.4 | 26.5 | 21.7 |
| length (cm) | | | |
| Fruiting truss | Prostrate | Erect | Erect |
| attitude | | | |
| Fruiting truss length | Medium | Long | Long |
| Fruit length (cm) | 4.93 | 3.76 | 4.35 |
| Fruit width (cm) | 4.36 | 3.73 | 3.92 |
| Fruit length/ | 1.13 | 1.01 | 1.11 |
| width ratio | | | |
| Fruit weight (g) | 27.5 | 21.0 | 22.3 |
| Relative fruit size | Very large | Medium | Medium |
| Predominant | Almost | Conical | Conical |
| fruit shape | cylindrical | | |
| Difference in shape | Slight | None or very | None or |
| between primary & | | slight | very slight |
| secondary fruits | | | |
| Band without | Narrow | Absent or very | Absent or |
| achenes | | narrow | very narrow |
| Unevenness of | Medium | Medium | Medium |
| fruit surface | | | |
| Fruit skin color | RHS 45B | RHS 46A | RHS 46B |
| | Red | Red | Red |
| Evenness of | Uneven | Even | Even |
| fruit color | | | |
| Fruit glossiness | Medium | Medium | Medium |
| Insertion of achenes | Level with | Level with | Level with |
| | surface | surface | surface |
| Achene coloration- | RHS 165A | RHS 166B | RHS 163B |
| sunward side of | Red to purple, | Red to purple, | Red to |
| berry | possibly greyed | possibly greyed | purple, |
| | | | possibly |
| | | | greyed |
| Achene coloration- | RHS 151C yellow | RHS 151C | RHS 151A |
| shaded side of berry | to green | yellow to green | Yellow to |
| | | | to green |
| Achenes per berry | 243.6 | 316.2 | 361.3 |
| Achene weight | 0.000664409 | 0.000570367 | 0.000466731 |
| Insertion of calyx | Set above | Level | Level |
| | the fruit | | |
| Pose of calx | Reflexed | Reflexed | Reflexed |
| segments | | | |
| Size of calyx in | Smaller | Same size | Same size |
| relation to fruit | to larger | | |
| Adherence of calyx | Strong | Strong | Strong |
| Firmness of flesh | Firm | Firm | Firm |
| Color of the flesh | RHS 155A | RHS 155A | RHS 155A |
| | (White) to RHS | (White) to RHS | (White) to RHS |
| | 44D (light red) | 44D (light red) | 38B (pale rose) |
| Evenness of | Slightly uneven | Even | Even |
| flesh color | | | |
| Distribution of | Marginal and | Only marginal | Only marginal |
| flesh color | central | | |
| Hollow center | Small | Absent | Medium |
| Sweetness | Strong | Medium | Medium |
| Acidity | Weak | Medium | Medium |
| Texture when tasted | Very fine | Medium | Medium |
| Time of flowering | Early | Early | Late |
| Harvest maturity | Mid-season, early | Mid-season, | Late |
| | June to late | early June to | early July to |
| | December | late December | late December |
| Type of bearing | Fully | Fully | Fully |
| | everbearing | everbearing | everbearing |
| Grams of fruit/plant | 500 | 286 | 289 |

Table 8 shows the resistance to different forms of stress of the new cultivar compared to ‘Driscoll Camarillo’ and ‘Baeza’. These forms of stress include drought, high temperatures, wind, high pH, and high soil salt level.

TABLE 8

| Reaction to | | ‘Driscoll | |
|---|---|---|---|
| Stress | ‘DrisStrawThree’ | Camarillo’ | ‘Baeza’ |
| Drought | Moderately | Moderately | Moderately |
| | resistant | resistant | resistant |
| High | Moderately | Moderately | Moderately |
| temperatures | resistant | resistant | resistant |
| Wind | Moderately | Moderately | Moderately |
| | resistant | resistant | resistant |
| High pH | Moderately | Moderately | Moderately |
| | resistant | resistant | resistant |
| High soil | Moderately | Moderately | Susceptible |
| salt level | resistant | resistant | |

Table 9 shows pest and disease characteristics of the new cultivar compared to ‘Driscoll Camarillo’ and ‘Baeza’.

TABLE 9

| | | ‘Driscoll | |
|---|---|---|---|
| Pest or Disease | ‘DrisStrawThree’ | Camarillo’ | ‘Baeza’ |
| Tetranychus | Moderately | Moderately | Moderately |
| urticae (2- | | | |
| spotted | resistant | resistant | resistant |
| spider mite) | | | |
| Tarsonemus | Moderately | Moderately | Moderately |
| pallidus | resistant | resistant | resistant |
| Aphelencoides | Moderately | Moderately | Moderately |
| fragariae | resistant | resistant | resistant |
| Pratylenchus | Moderately | Moderately | Moderately |
| penetrans | resistant | resistant | resistant |
| Ditylenchus | Moderately | Moderately | Moderately |
| dipsac | resistant | resistant | resistant |
| Anthonomus | Moderately | Moderately | Moderately |
| rubi | resistant | resistant | resistant |
| Aphis spp. | Moderately | Moderately | Moderately |
| (Aphids) | resistant | resistant | resistant |
| Lygus hesperus | Moderately | Moderately | Moderately |
| (Lygus bug) | resistant | resistant | resistant |
| Botrytis | Moderately | Moderately | Moderately |
| fruit rot | Susceptible | Susceptible | Susceptible |
| Powdery | Resistant | Highly | Susceptible |
| mildew | | susceptible | |

We claim:

1. A new and distinct cultivar of strawberry plant as described and shown herein.
